(19) United States
(12) Patent Application Publication
(10) Pub. No.: US 2026/0279523 A1
(43) Pub. Date: Sep. 17, 2026

(54) SYSTEM AND METHOD FOR AI-ASSISTED RELATIONSHIP JOURNALING AND SENTIMENT ANALYSIS

(71) Applicant: Hill Bryant Abrahams, Tallahassee, FL (US)

(72) Inventor: Hill Bryant Abrahams, Tallahassee, FL (US)

(21) Appl. No.: 19/355,119

(22) Filed: Oct. 10, 2025

Related U.S. Application Data
(60) Provisional application No. 63/771,764, filed on Mar. 14, 2025.

(51) Int. Cl. G16H 10/60; G06F 16/18; G06F 21/31; G06F 40/30

(52) U.S. Cl. CPC G16H 10/60; G06F 16/1815; G06F 21/31; G06F 40/30; G06F 2221/2117

(57) ABSTRACT

A computerized system and method for implementing the UCANRR (Understanding the Care, Abuse, and Neglect Recognition and Reporting) psychological model in relationship therapy. The system comprises a subscription-based web platform for therapist registration, mobile applications for client journaling, AI-powered sentiment analysis engines, and comprehensive reporting tools. Journal entries are analyzed using natural language processing to assign numerical scores on a Care-Neglect-Abuse continuum ranging from +10 to −10, with special detection and reinforcement of repair attempts. Therapists access scatter plot visualizations showing sentiment trends over time while client journal privacy is maintained through local-only storage of entry text. The system provides quantifiable, data-driven insights to complement traditional therapeutic approaches in couples counseling.

### CROSS-REFERENCE TO RELATED APPLICATION

This application claims the benefit of U.S. Provisional Patent Application No. 63/771,764, filed Mar. 14, 2025, entitled “NOVEL PSYCHOLOGICAL MODEL FOR JOURNALING USING A COORDINATED WEBSITE AND MOBILE APPLICATION SOLD AS A SUBSCRIPTION TO PROFESSIONAL PSYCHOLOGISTS,” the entire contents of which are incorporated herein by reference.

### FIELD OF THE INVENTION

The present invention relates generally to computerized therapeutic journaling systems, and more particularly to a subscription-based platform implementing the UCANRR (Understanding the Care, Abuse, and Neglect Recognition and Reporting) psychological model for quantifying relationship dynamics through AI-powered sentiment analysis and therapist-monitored visualization.

### BACKGROUND OF THE INVENTION

Traditional relationship therapy relies heavily on subjective client recall during periodic therapy sessions, often conducted weekly or biweekly. Between sessions, significant relationship events occur that may be forgotten, misremembered, or minimized by the time the next therapeutic appointment arrives. This creates several problems for effective therapeutic intervention.

First, the accuracy of client recall degrades over time. emotions that were intense

during an actual interaction may seem less significant days later, or conversely, minor incidents may be amplified through rumination. Second, partners in a relationship may remember the same event very differently, leading to therapeutic sessions dominated by “he said, she said” disputes rather than productive analysis. Third, therapists lack quantifiable data to track relationship progress or deterioration over time, making it difficult to objectively measure therapeutic outcomes.

Existing journaling applications provide generic platforms for recording thoughts and feelings but lack specialized frameworks for relationship therapy. Current mental health applications may include mood tracking or general sentiment analysis, but these systems do not provide:
A structured psychological model specifically designed for relationship dynamics
Dyadic tracking that links partners in therapy together
Therapist-accessible reporting with quantifiable metrics
AI-powered sentiment analysis calibrated to relationship-specific language
Visual trend analysis showing patterns of care, neglect, and abuse over time
Recognition and reinforcement of repair attempts as distinct therapeutic events

Moreover, conventional systems do not integrate client journaling with professional therapeutic practice in a HIPAA-compliant, secure manner that preserves client privacy while providing therapists with actionable insights.

The UCANRR system addresses these deficiencies by implementing a novel psychological framework that categorizes relationship interactions along a Care-Neglect-Abuse continuum, with numerical scoring from +10 (maximum care) through 0 (neglect) to −10 (maximum abuse). The system further recognizes repair attempts as critically important relationship events deserving special tracking and positive reinforcement.

### SUMMARY OF THE INVENTION

The present invention provides a computerized system and method for implementing the UCANRR (Understanding the Care, Abuse, and Neglect Recognition and Reporting) psychological model in relationship therapy. The system comprises a web-based subscription service for professional therapists, coordinated mobile applications for client journaling, AI-powered sentiment analysis engines, and comprehensive reporting tools for therapist review.

In one aspect, the invention provides a relationship therapy system comprising: a server hosting a subscription-based web application for therapist registration and client management; mobile applications executing on client devices for secure journal entry creation; an AI sentiment analysis engine that processes journal entry text and assigns numerical scores on a Care-Neglect-Abuse continuum ranging from +10 to −10; a relational database linking therapist accounts to client pairs and storing sentiment scores with temporal metadata; and a reporting interface generating visual representations of relationship trends over time.

In another aspect, the invention provides a method for quantifying relationship dynamics, comprising: enrolling a therapist via web-based subscription; registering a client pair associated with the therapist; receiving text-based journal entries from clients via mobile applications; processing each journal entry through AI sentiment analysis to generate a numerical score on a −10 to +10 scale; identifying repair attempts and applying positive weighting; storing sentiment scores with timestamps in a therapist-accessible database while maintaining journal entry privacy; and generating scatter plot visualizations with trend lines showing relationship patterns over time.

In a further aspect, the invention provides a computer-readable storage medium containing instructions that, when executed by a processor, implement the UCANRR psychological model by: accepting journal entry text; parsing the text into semantic units; applying natural language processing to identify emotional valence; mapping detected sentiment to the Care-Neglect-Abuse continuum; detecting repair attempt language patterns; assigning numerical scores with repair bonuses; and transmitting scores to a therapist reporting system.

The UCANRR system distinguishes itself from conventional therapy tools through its novel psychological framework, dyadic client pairing, optional partner sharing via SMS, repair attempt recognition, and AI-calibrated sentiment scoring specifically designed for relationship dynamics.

These and other features, aspects and advantages of the present invention will become better understood with reference to the following drawings, description and claims.

### BRIEF DESCRIPTION OF THE DRAWINGS

The invention will be better understood from the following detailed description with reference to the drawings, in which:

FIG. 1 is a system architecture diagram showing the interconnection between web server, therapist interface, client mobile devices, and client users in the UCANRR system;

FIG. 2 is a process flowchart illustrating the operational steps from therapist registration through client journaling to therapeutic reporting;

FIG. 3 is a database schema diagram showing the relational structure of THERAPIST, CLIENT, and EVENT tables with their respective fields and relationships;

FIG. 4 is a screenshot of the mobile application welcome screen displaying the PsychInsightPro interface with an “Analyze your journal entries with AI-driven insights” tagline and Get Started button;

FIG. 5 is a screenshot of the mobile application main menu showing New Entry, Reports, and Setup Share Phone Number buttons;

FIG. 6 is a screenshot of the journal entry interface with a text input field for entering journal content and an “Analyze and Save” button;

FIG. 7 is a screenshot showing AI sentiment analysis results for a neglect-related journal entry, displaying Score: −2, reasoning explaining emotional neglect detection, a confidence level of 0.8, and a Share button;

FIG. 8 is a screenshot showing AI sentiment analysis results for an abuse-related journal entry, displaying Score: −3, reasoning explaining detection of sarcastic/humiliating behavior, a confidence level of 0.9, and a Share button;

FIG. 9 is a screenshot showing AI sentiment analysis results for a care-related journal entry, displaying Score: 2, reasoning explaining detection of positive supportive interaction, a confidence level of 0.9, and a Share button;

FIG. 10 is a screenshot of the therapist reporting interface showing date range selection, a sentiment analysis scatter plot with trend line, and summary statistics including Neglect Count, Repair Count, Shared Count, and Bid Count; In color, scatter points >0 are green, else red. Positive slope of trend line is green, else red; and

FIG. 11 is a screenshot of the client reporting interface with a drill-down popup displaying the full journal entry text associated with a selected data point on the scatter plot. In color on FIGS. 10 and 11, selected counts for Neglect, Repair, Shared and Bid along with data points are highlighted in yellow.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

### System Architecture Overview

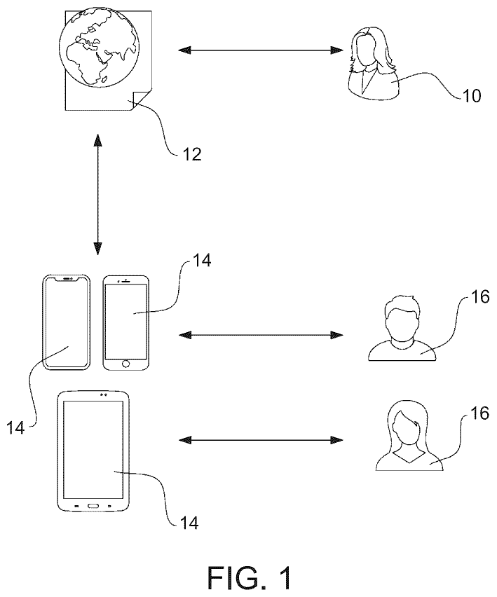

Referring to FIG. 1, the UCANRR system architecture comprises a web server 10 hosting the subscription service and therapist interface, a therapist user 12 accessing the web-based platform, multiple client mobile devices 14 executing iOS and Android applications, and client users 16 who are partners in therapy. The web server 10 maintains secure encrypted communications with both therapist workstations and client mobile devices 14 via HTTPS protocols.

The system implements a three-tier architecture: (1) presentation layer comprising web and mobile user interfaces; (2) application layer containing business logic for user authentication, journal processing, and AI sentiment analysis; and (3) data layer consisting of relational databases storing therapist accounts, client records, and sentiment-scored events.

### UCANRR Psychological Model

The Care, Abuse, and Neglect (CAN) Continuum represents a novel psychological framework for categorizing relationship interactions. Unlike binary classification systems that simply label behaviors as “good” or “bad,” the UCANRR model recognizes that relationship dynamics exist along a spectrum with three distinct zones:

Care Zone (+1 to +10): Interactions demonstrating love, empathy, support, appreciation, affection, active listening, validation, emotional availability, and constructive communication. Higher scores indicate more profound expressions of care, such as sacrificial acts of service (+8 to +10), while lower positive scores represent routine kindness and consideration (+1 to +3).

Neglect Zone (0): Interactions characterized by emotional unavailability, lack of response, disengagement, avoidance, stonewalling, and absence of recognition. Neglect represents neither active harm nor active care, but rather a void in emotional connection. The zero score reflects this neutral-negative state where relationship needs go unmet.

Abuse Zone (−1 to −10): Interactions involving harmful behaviors including verbal attacks, criticism, contempt, emotional manipulation, gaslighting, threats, and physical aggression. Mild abuse (−1 to −3) might include sarcasm or dismissiveness, while severe abuse (−8 to −10) involves overtly destructive or violent behavior.

Repair Attempts: The UCANRR model recognizes repair attempts as a distinct category of relationship behavior deserving special attention. Repair attempts are efforts by one partner to de-escalate conflict, apologize, take responsibility, seek reconciliation, or otherwise mend relational damage. When detected, repair attempts receive a +2 bonus modifier added to the base sentiment score, and are marked with a special indicator in therapist reports. This positive reinforcement encourages clients to recognize and value their own repair efforts.

### Operational Process Flow

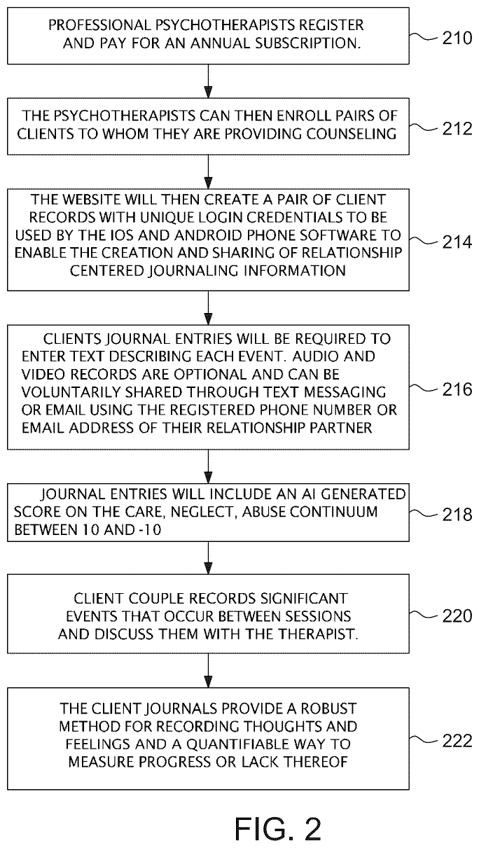

Referring to FIG. 2, the operational flow begins at step 210 where professional psychotherapists register and pay for an annual subscription via the web-based platform. Registration includes credential verification, payment processing, and account setup with unique therapist identification numbers.

At step 212, psychotherapists enroll pairs of clients to whom they are providing counseling. Each client pair represents a romantic partnership, married couple, or other dyadic relationship undergoing therapy. The system creates linked client records with distinct login credentials.

At step 214, the website creates a pair of client records with unique login credentials to be used by the iOS and Android phone software to enable the creation and sharing of relationship-centered journaling information. Each client receives installation instructions for their respective mobile device platform and individualized login credentials.

At step 216, clients' journal entries are required to enter text describing each significant relationship event. Audio and video records are optional and can be voluntarily shared through text messaging or email using the registered phone number or email address of their relationship partner. The primary journaling modality is text-based to facilitate AI sentiment analysis.

At step 218, journal entries include an AI-generated score on the Care, Neglect, Abuse continuum between +10 and −10. The sentiment analysis occurs in real-time or near-real-time, providing immediate feedback to the client about how their reported interaction is characterized by the UCANRR model.

At step 220, therapist and client couple record significant events that occur between sessions and discuss them during therapeutic appointments. The therapist accesses quantified data showing patterns of interaction over time.

At step 222, the client journals provide a robust method for recording thoughts and feelings and a quantifiable way to measure progress or lack thereof. The system generates objective metrics that complement subjective therapeutic discussions.

### Database Structure

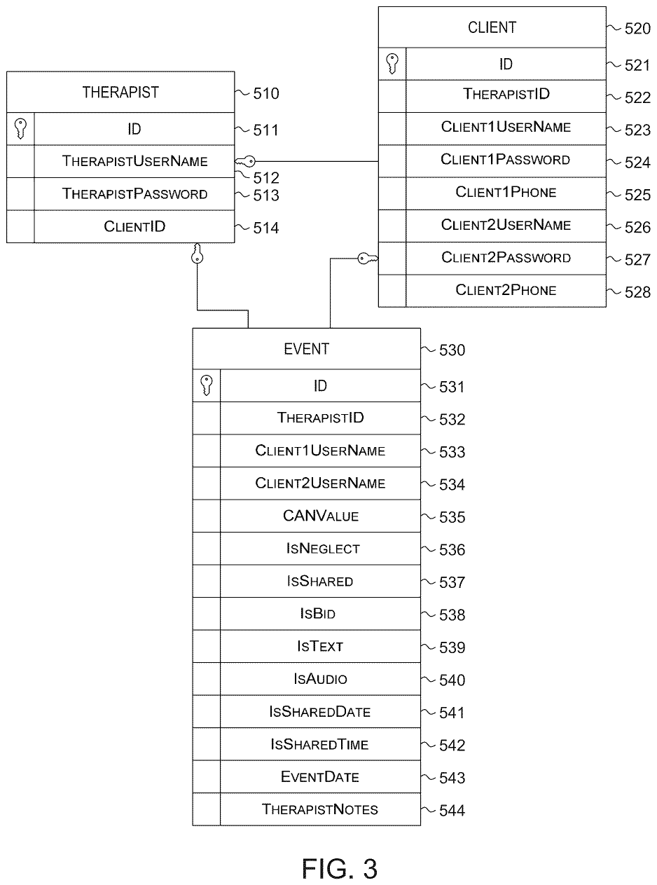

Referring to FIG. 3, the relational database structure comprises three primary tables: THERAPIST 510, CLIENT 520, and EVENT 530.

The THERAPIST table 510 includes fields for:
ID 511: Unique therapist identifier (primary key)
THERAPISTUSERNAME 512: Login credential
THERAPISTPASSWORD 513: Encrypted authentication credential
CLIENTID 514: Foreign key linking to client pairs

The CLIENT table 520 includes fields for:
ID 521: Unique client identifier (primary key)
THERAPISTID 522: Foreign key linking to therapist
CLIENT1USERNAME 523: First partner's login credential
CLIENT1PASSWORD 524: Encrypted authentication credential
CLIENT1PHONE 525: Contact information for SMS sharing
CLIENT2USERNAME 526: Second partner's login credential
CLIENT2PASSWORD 527: Encrypted authentication credential
CLIENT2PHONE 528: Contact information for SMS sharing

The EVENT table 530 includes fields for:
ID 531: Unique event identifier (primary key)
THERAPISTID 532: Foreign key linking to therapist
CLIENT1USERNAME 533: Identifier for journal entry author
CLIENT2USERNAME 534: Identifier for partner
CANVALUE 535: Sentiment score (−10 to +10)
ISNEGLECT 536 Boolean flag for Neglect detected
ISSHARED 537: Boolean flag indicating if entry was shared with partner
ISBID 538 Boolean flag for Bid detected
ISTEXT 539: Boolean flag for text entry type
ISAUDIO 540: Boolean flag for audio attachment
ISSHAREDDATE 541: Timestamp of sharing event
ISSHAREDTIME 542: Time of sharing event
EVENTDATE 543: Timestamp of journal entry creation
THERAPISTNOTES 544: Optional therapist annotations

This relational structure enables efficient querying for therapist reports while maintaining separation between client-private journal content (stored locally on mobile devices) and therapist-accessible sentiment metadata (stored on server).

### Mobile Application Interface

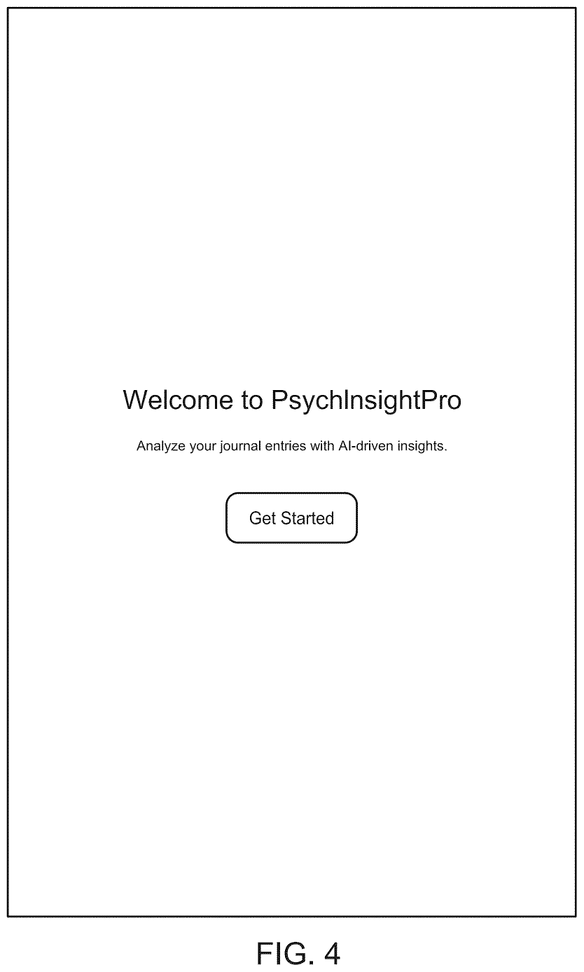

Referring to FIGS. 4-9, the mobile application provides an intuitive interface for client journaling and sentiment feedback.

FIG. 4 shows the welcome screen displaying “Welcome to Psychinsightpro” with the tagline “Analyze your journal entries with AI-driven insights” and a “Get Started” button for first-time users.

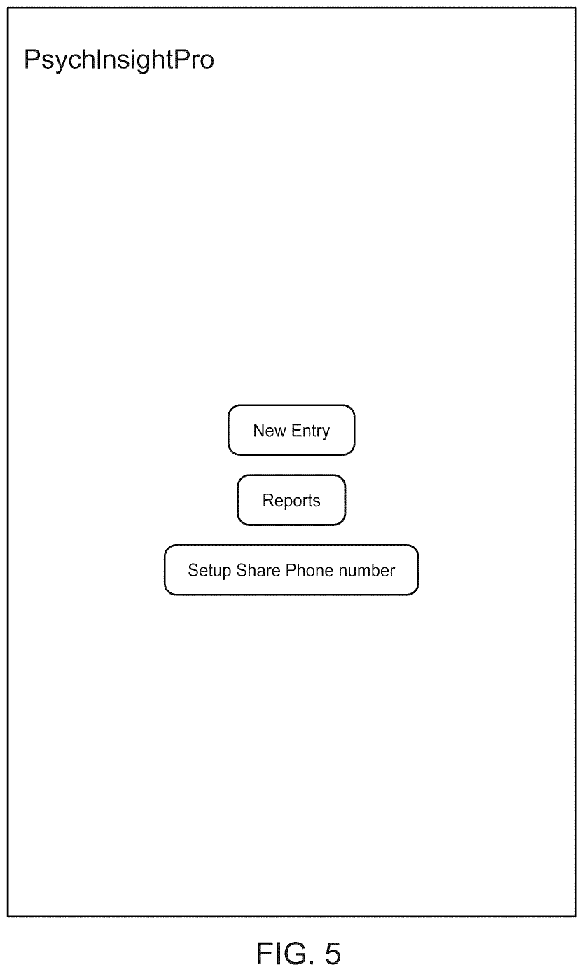

FIG. 5 depicts the main menu with three primary functions: “New Entry” button for creating journal entries, “Reports” button for viewing personal sentiment trends, and “Setup Share Phone number” button for configuring optional partner sharing via SMS.

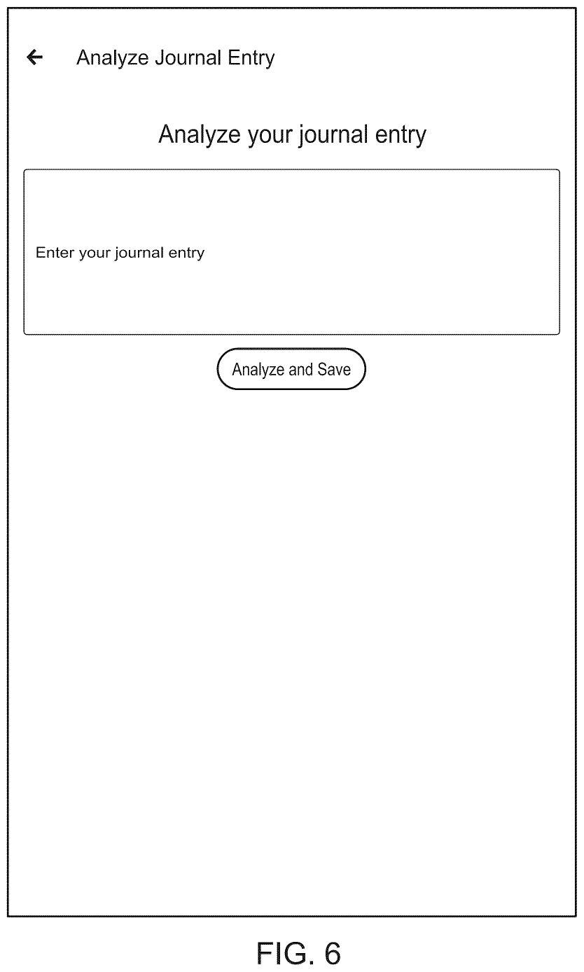

FIG. 6 illustrates the journal entry interface with a text input field labeled “Enter your journal entry” and an “Analyze and Save” button. This interface encourages clients to describe relationship events in their own words without constraint.

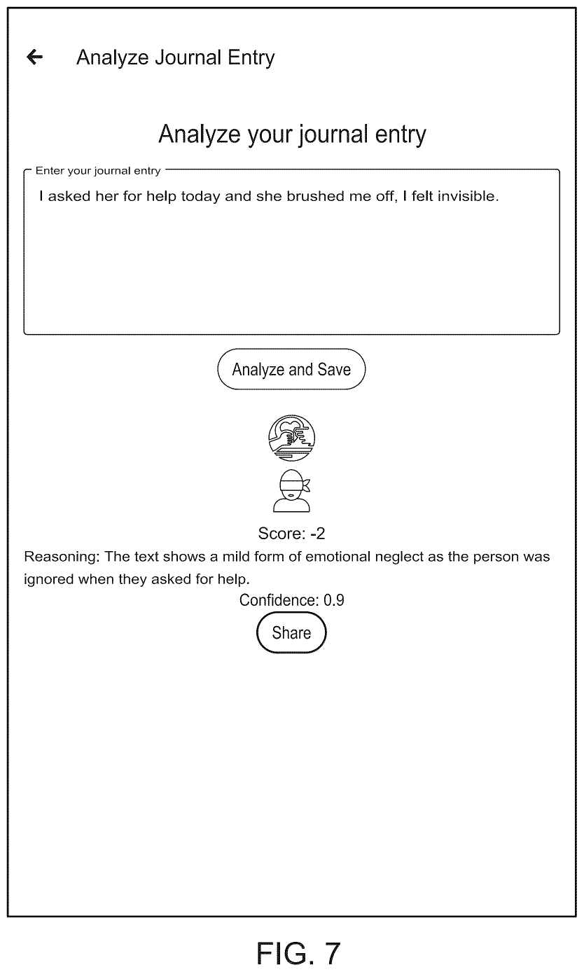

FIG. 7 demonstrates the AI sentiment analysis result for a neglect-related entry: “I asked her for help today and she brushed me off. I felt invisible.” The system displays Score: −2 with reasoning: “The text shows a mild form of emotional neglect as the person was ignored when they asked for help,” along with Confidence: 0.8 and a “Share” button for optional partner notification.

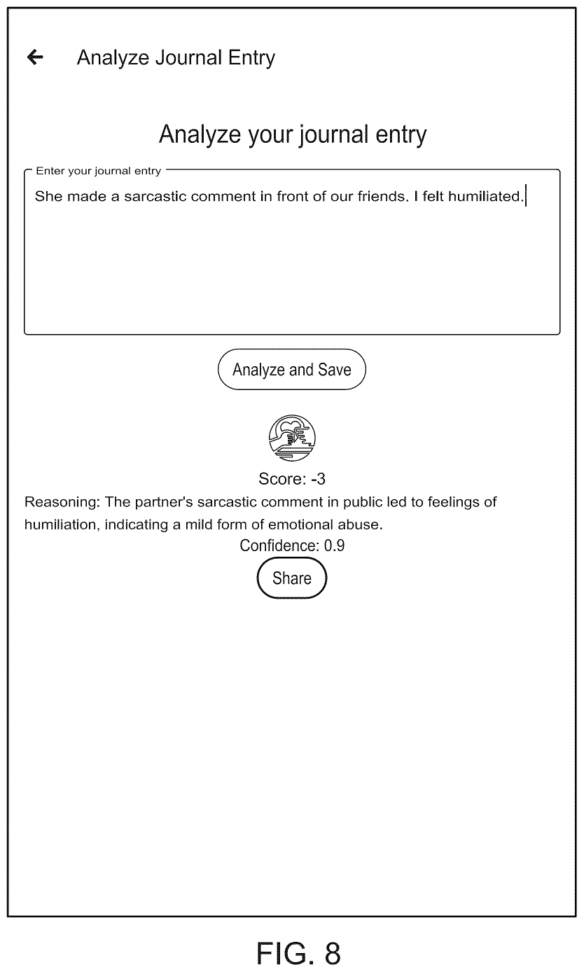

FIG. 8 shows analysis of an abuse-related entry: “She made a sarcastic comment in front of our friends. I felt humiliated.” The system displays Score: −3 with reasoning: “The partner's sarcastic comment in public led to feelings of humiliation, indicating a mild form of emotional abuse,” Confidence: 0.9, and a “Share” button.

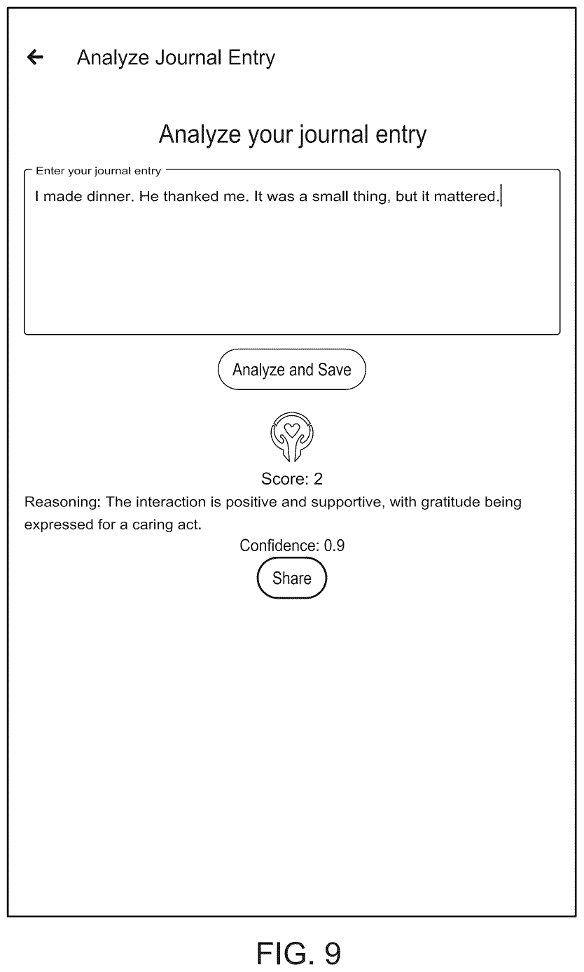

FIG. 9 presents analysis of a care-related entry: “I made dinner. He thanked me. It was a small thing, but it mattered.” The system displays Score: 2 with reasoning: “The interaction is positive and supportive, with gratitude being expressed for a caring act,” Confidence: 0.9, and a “Share” button.

The AI reasoning display serves multiple therapeutic functions: (1) it validates the client's emotional experience by acknowledging the significance of the interaction; (2) it educates clients about the UCANRR framework by explaining how their experience maps to the Care-Neglect-Abuse continuum; and (3) it provides transparency in the AI scoring process, building trust in the system.

### Therapist and Client Reporting Interfaces

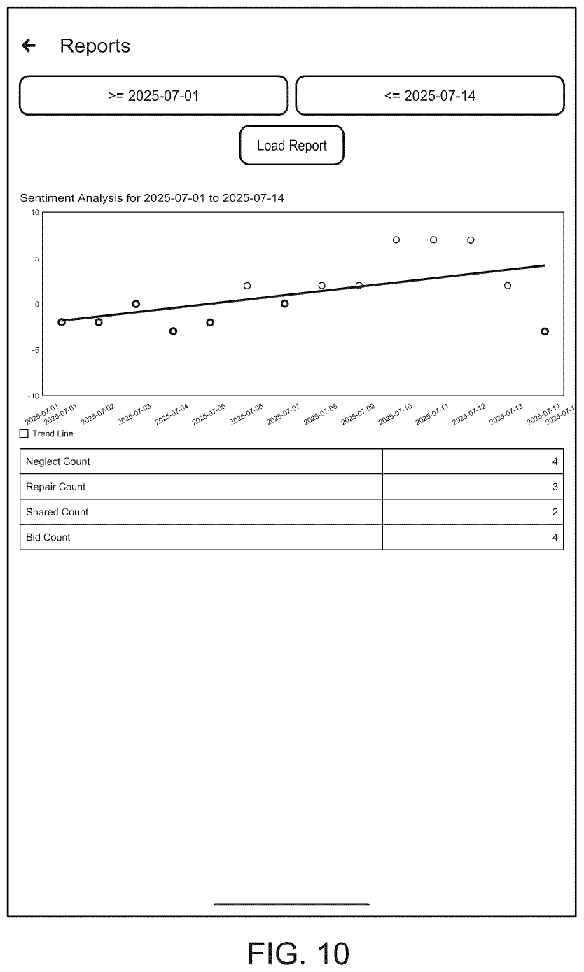

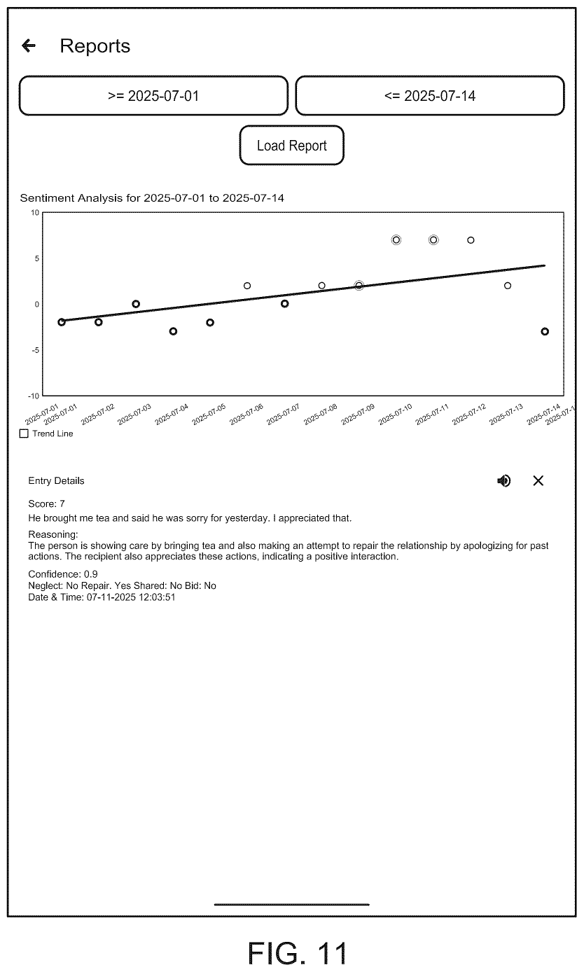

Referring to FIGS. 10-11, the therapist reporting interface provides comprehensive visualization and analysis tools.

FIG. 10 shows the reports dashboard with date range selection fields (“>=Jul. 1, 2025” and “<=Jul. 14, 2025”), a “Load Report” button, and a “Sentiment Analysis” scatter plot displaying sentiment scores over time with a linear trend line. Data points are plotted chronologically on the x-axis with sentiment scores on the y-axis. The interface includes summary statistics: Neglect Count: 4, Repair Count: 3, Shared Count: 3, Bid Count: 4. These metrics provide therapists with quick quantitative assessments of relationship patterns. In color positive scatter plot data points are green, else red. Positive slope of trend line is green, else red.

FIG. 11 demonstrates the drill-down functionality where clicking a data point on the scatter plot opens a popup window displaying the complete journal entry text on the client device. In this example, the entry shows Score: 0 with the text: “He stayed out late without calling. It's like I don't even exist.” The popup includes audio/speaker icons indicating a text reader is available, a close (X) button, and contact information (“Date & Time: July 14 12:01:01”). This drill-down capability allows clients to move seamlessly between quantitative trend analysis and qualitative journal content review.

### AI Sentiment Analysis Engine

The AI sentiment analysis engine implements natural language processing (NLP) algorithms specifically trained on relationship-relevant language patterns. The analysis process comprises several stages:

Text Preprocessing: Journal entries undergo tokenization, removing stop words while preserving emotionally significant terms. Punctuation and capitalization patterns are analyzed for emotional intensity indicators (e.g., all-caps text suggesting anger).

Semantic Parsing: The text is divided into semantic units (sentences or clauses) representing discrete thoughts or events. Each semantic unit is analyzed independently before aggregation.

Sentiment Classification: Each semantic unit is classified using a combination of lexicon-based methods and machine learning models. The system maintains a specialized lexicon of relationship-relevant terms with pre-assigned sentiment values. Examples include:
Care terms: “love,” “appreciate,” “listen,” “support,” “thank,” “help,” “comfort” (+2 to +5)
Neglect terms: “ignore,” “brush off,” “dismiss,” “unavailable,” “silent treatment” (−1 to −2)
Abuse terms: “yell,” “insult,” “threaten,” “control,” “manipulate,” “hit” (-4 to-10)

Contextual Weighting: The system applies contextual analysis to adjust scores based on surrounding language. For example, “I felt heard” increases positive weighting, while “I felt invisible” increases negative weighting. Negation detection ensures that “he did not ignore me” is properly scored as positive rather than negative.

Repair Detection: the system identifies repair attempts through specific linguistic markers including:
Apology language: “I'm sorry,” “I apologize,” “my fault”
Reconciliation efforts: “let's talk,” “can we work this out,” “I want to fix this”
Taking responsibility: “I was wrong,” “I understand why you're upset”
Soothing behaviors: “I love you,” “we'll get through this,” “you matter to me”

When repair language is detected, the system adds +2 to the base sentiment score and flags the entry with a repair marker for special visibility in therapist reports.

Confidence Scoring: Each sentiment analysis includes a confidence score (0.0 to 1.0) reflecting the AI's certainty in its classification. Low confidence scores may indicate ambiguous language, sarcasm, or complex emotional content requiring human interpretation. Therapists can review low-confidence entries with particular attention during sessions.

Score Aggregation: For multi-sentence journal entries, individual sentence scores are aggregated using weighted averaging. More emotionally intense sentences receive higher weighting in the final score calculation.

Privacy and Security Features The UCANRR system implements multiple layers of privacy protection:

Client Journal Privacy: The full text of journal entries remains stored locally on client mobile devices 14. Only sentiment scores, timestamps, and metadata are transmitted to the server 10. This architecture ensures that therapists cannot access raw journal content unless clients explicitly choose to share entries during therapy sessions or via the SMS sharing function.

Encryption: All data transmissions between mobile devices 14 and server 10 utilize TLS 1.3 encryption. Passwords are stored using bcrypt hashing with salt. Database records are encrypted at rest using AES-256 encryption.

HIPAA Compliance: The system architecture is designed to comply with Health Insurance Portability and Accountability Act (HIPAA) requirements for protected health information (PHI). Access controls, audit logging, and data retention policies follow HIPAA technical safeguards.

Optional Sharing Controls: The SMS sharing feature (FIGS. 7-9) gives clients complete control over whether to share specific journal entries with their partner. Sharing is never automatic or required. When a client chooses to share an entry, only the client's own entry is sent—not the partner's entries. The fact that sharing occurred (timestamp and sentiment score) is logged in the therapist's report for discussion purposes.

### Therapist Subscription and Client Management

The web-based platform enables therapists to manage multiple client pairs simultaneously. After subscribing, therapists can:

Add Client Pairs: Enter identifying information for both partners, generate unique login credentials, and link the pair to the therapist's account.

View Reports: Access sentiment analysis data for any enrolled client pair, with customizable date ranges for trend analysis.

Annotate Events: Add therapist notes (field 544 in FIG. 3) to specific journal events for session planning and progress tracking.

Track Engagement: Monitor client journaling frequency and compliance with between-session homework assignments.

Manage Subscriptions: Renew annual subscriptions, add additional client pair slots, and update billing information.

The subscription model provides therapists with a predictable annual cost structure while enabling unlimited journal entries per client pair. This removes financial barriers to client engagement and encourages frequent journaling.

### Repair Attempt Visualization

In therapist reports (FIGS. 10-11), repair attempts are marked with distinctive visual indicators, such as a yellow border when Repair Count is clicked on the scatter plot. This allows therapists to quickly identify when clients are making reconciliation efforts. The trend line analysis can show whether repair attempts, care, neglect, and abuse are increasing or decreasing over time, providing objective data about relationship health trajectories.

During therapy sessions, therapists can use repair markers as discussion points: “I notice you made three repair attempts last week. Let's talk about how those efforts were received by your partner.” This data-driven approach grounds therapeutic discussions in concrete behavioral evidence rather than subjective memory.

### Future Enhancements and Adaptations

While the primary embodiment focuses on couples therapy, the UCANRR system can be adapted for additional use cases:

Solo Therapy: Individual clients can use the system to track relationship patterns with various people in their lives (family members, friends, coworkers, and themselves) without requiring partner enrollment. The sentiment analysis and trend visualization remain valuable for personal insight and therapeutic discussion.

Group Therapy: Multiple client accounts can be linked to a single therapist, enabling group dynamics analysis and comparative trend visualization.

Voice and Video Analysis: Future implementations may incorporate voice tone analysis and facial expression recognition to provide multimodal sentiment assessment beyond text-based journaling.

Predictive Analytics: Machine learning models can be trained on aggregate sentiment data to identify early warning patterns predictive of relationship crisis, enabling proactive therapeutic intervention.

### Hardware Standardization (Optional Implementation)

In some embodiments, particularly during research validation studies, therapists receive standardized hardware packages to ensure consistent user experience and optimal system performance. Such packages may include:
Large-screen all-in-one computers (24-27 inches) for enhanced data visualization
High-resolution displays supporting detailed scatter plot analysis
Pre-configured software environments with optimized browser settings
AI-enabled cameras for future video analysis features
Ergonomic input devices (keyboard and mouse)

Hardware standardization reduces technical support burden, ensures HIPAA-compliant device configuration, and provides consistent visualization quality across all therapist users. In production deployments, therapists may purchase standardized packages with flexible payment plans or alternatively use their own hardware meeting minimum system requirements.

### Alternative Embodiments

The invention encompasses various alternative implementations:

Alternative Scoring Scales: While the primary embodiment uses a −10 to +10 scale, alternative implementations might use 0-100 scales, letter grades (A-F), or color-coded categories (green-yellow-red).

Alternative AI Models: The sentiment analysis engine can be implemented using various NLP architectures including transformer-based models (BERT, GPT), recurrent neural networks (LSTM), or ensemble methods combining multiple algorithms.

Alternative Platforms: While the primary embodiment describes iOS and Android mobile applications, the system can be implemented as a progressive web application (PWA), desktop software, or browser-based interface accessible across multiple platforms.

Alternative Therapist Interfaces: The web-based therapist dashboard can be supplemented or replaced with dedicated desktop applications, mobile apps for therapist use, or integrated electronic health record (EHR) system plugins.

It should be understood, of course, that the foregoing relates to exemplary embodiments of the invention and that modifications may be made without departing from the spirit and scope of the invention as set forth in the following claims.

What is claimed is:

1. A computerized system for quantifying relationship dynamics in therapeutic settings, comprising:
a processor executing instructions stored in non-transitory memory;
a web-based subscription service hosted on a server and accessible to licensed therapists for account registration and client management;
a relational database stored on the server, the database configured to store:
therapist account records comprising unique therapist identifiers and authentication credentials;
client pair records comprising first and second client identifiers linked to a therapist identifier, wherein each client pair represents partners in a therapeutic relationship; and
event records comprising sentiment scores, timestamps, and metadata associated with journal entries created by clients in the client pair;
a mobile application executing on client mobile devices, the mobile application comprising:
a user interface configured to receive text-based journal entries describing relationship interactions;
a transmission module configured to securely transmit journal entry text to an AI sentiment analysis engine; and
a display module configured to present sentiment analysis results to the client; and
a local database on the client device for storing text entries, score, reasoning, confidence, neglect, repair, shared and bid indicators, date and time
an AI sentiment analysis engine configured to:
receive journal entry text from the mobile application;
process the journal entry text using natural language processing algorithms to identify emotional valence;
assign a numerical sentiment score to the journal entry based on a Care-Neglect-Abuse continuum, wherein care interactions receive positive scores ranging from +1 to +10, neglect interactions receive a score of 0, and abuse interactions receive negative scores ranging from −1 to −10;
detect repair attempt language patterns within the journal entry text; and
apply a positive modifier to the sentiment score when repair attempt language is detected; and
a therapist reporting interface accessible via the web-based subscription service, the reporting interface configured to:
retrieve event records from the relational database for a selected client pair and date range;
generate a visual scatter plot displaying sentiment scores plotted against timestamps including color code green for positive scores (Care), otherwise red;
overlay a trend line on the scatter plot to illustrate sentiment patterns over time where a positive slope is color coded green, else red.

2. A method for implementing a psychological model for relationship therapy, comprising:
enrolling a licensed therapist by:
receiving therapist registration information via a web-based interface;
processing subscription payment for annual access to a therapy platform;
creating a therapist account record in a relational database with unique authentication credentials; and
granting the therapist access to a reporting dashboard; registering a client pair by:
receiving identification information for a first client and a second client from the therapist;
generating unique login credentials for the first client and the second client;
creating client records in the relational database linked to the therapist account record; and
providing mobile application installation instructions and login credentials to the first client and the second client;
receiving journal entries by:
accepting text input via a mobile application interface executing on a client mobile device, wherein the text input describes a relationship interaction between the first client and the second client;
transmitting the text input to a sentiment analysis engine hosted on a remote server;
analyzing sentiment by:
parsing the text input into semantic units using natural language processing;
identifying emotional valence of each semantic unit based on a lexicon of relationship-relevant terms;
classifying the text input as representing care, neglect, or abuse based on the identified emotional valence;
assigning a numerical score on a scale ranging from −10 to +10, wherein positive scores indicate care, zero indicates neglect, and negative scores indicate abuse;
detecting presence of repair attempt language within the text input;
applying a +2 modifier to the numerical score when repair attempt language is detected;
generating a confidence score indicating certainty of the sentiment classification; and
transmitting the numerical score, confidence score, and reasoning explanation back to the mobile application;
storing event data by:
creating an event record in the relational database comprising the numerical score, a timestamp, and metadata identifying the client pair;
maintaining the full text of the journal entry locally on the client mobile device without transmitting the full text to the remote server;
generating reports by:
receiving a request from the therapist via the reporting dashboard to view sentiment data for the client pair;
querying the relational database for event records associated with the client pair within a specified date range;
calculating summary statistics comprising counts of neglect events, repair attempt events, shared entries and bid attempts;
generating a scatter plot visualization displaying the numerical scores plotted chronologically and color coded; green for positive scores (Care), otherwise red;
overlaying a trend line on the scatter plot using linear regression or moving average calculation with positive slope color coded green, otherwise red; and
presenting the scatter plot and summary statistics to the therapist via the reporting dashboard.

3. A non-transitory computer-readable storage medium containing instructions that, when executed by a processor, cause the processor to implement a Care-Neglect-Abuse continuum psychological model by:
receiving journal entry text via an application programming interface (API),
wherein the journal entry text comprises a narrative description of a relationship interaction;
tokenizing the journal entry text into a plurality of words and phrases;
removing stop words from the plurality of words and phrases to generate a set of semantically significant terms;
comparing the set of semantically significant terms against a sentiment lexicon comprising:
care-related terms associated with positive numerical values ranging from +1 to +10;
neglect-related terms associated with a zero numerical value; and
abuse-related terms associated with negative numerical values ranging from −1 to −10;
calculating a preliminary sentiment score by aggregating numerical values associated with matching terms from the sentiment lexicon;
analyzing contextual information surrounding the matching terms to adjust the preliminary sentiment score, wherein the contextual information comprises:
negation words that reverse the polarity of adjacent terms;
intensifier words that amplify the magnitude of adjacent terms; and
emotion words that indicate subjective emotional states;
searching the journal entry text for repair attempt indicators comprising:
apology language patterns;
reconciliation effort phrases;
responsibility-taking statements; and
soothing behavior expressions;
when one or more repair attempt indicators are detected:
adding a +2 modifier to the preliminary sentiment score to generate a final sentiment score; and
setting a repair attempt flag to true;
when no repair attempt indicators are detected:
designating the preliminary sentiment score as the final sentiment score; and
setting the repair attempt flag to false;
calculating a confidence score ranging from 0.0 to 1.0 based on:
strength of lexicon matches;
clarity of emotional language; and
absence of ambiguous or sarcastic phrasing;
generating a reasoning explanation describing:
which terms contributed to the final sentiment score;
whether care, neglect, or abuse was detected; and
whether a repair attempt was identified;
transmitting a response comprising the final sentiment score, the repair attempt flag, the confidence score, and the reasoning explanation via the application programming interface; and
storing the final sentiment score, the repair attempt flag, bid, shared, and neglect flags in a database record associated with a client identifier and a timestamp, wherein the journal entry text is not stored in the database record.

4. The system of claim 1, wherein the mobile application further comprises an optional sharing module configured to:
present a user interface element enabling the client to selectively share a journal entry with their partner via SMS messaging;
upon receiving user input activating the sharing function, transmit the journal entry text to a phone number associated with the partner; and
log the sharing event in the database by creating a sharing record comprising a timestamp, the sentiment score, and identifiers for both the sending client and the receiving partner, wherein the sharing record is accessible to the therapist via the reporting interface.

5. The system of claim 1, wherein the AI sentiment analysis engine is further configured to:
parse the journal entry text into a plurality of sentences;
assign individual sentiment scores to each sentence in the plurality of sentences;
identify a subset of sentences having the highest emotional intensity based on presence of intensifier words, capitalization patterns, or exclamation marks;
apply higher weighting to sentiment scores of the subset of sentences; and
calculate an aggregated sentiment score for the entire journal entry by computing a weighted average of the individual sentiment scores.

6. The system of claim 1, wherein the therapist reporting interface is further configured to:
display repair attempt markers on the scatter plot at data points corresponding to event records with detected repair attempts;
calculate a repair attempt frequency metric indicating a number of repair attempts per week or per month; and
display trend information comparing repair attempt frequency across different time periods.

7. The system of claim 1, wherein the relational database further comprises:
a therapist notes field associated with each event record, the therapist notes field configured to store annotations entered by the therapist via the reporting interface; and
wherein the therapist reporting interface is further configured to:
display an input field for entering therapist notes when viewing a specific journal entry detail; and
save the entered therapist notes to the therapist notes field in the
corresponding event record.

8. The method of claim 2, further comprising:
enabling optional partner sharing by:
receiving a phone number from the first client via the mobile application;
storing the phone number in the client record associated with the second client;
presenting a share button in the mobile application interface after displaying sentiment analysis results;
upon detecting selection of the share button, transmitting the journal entry text via SMS to the stored phone number; and
creating a sharing event record in the relational database comprising a timestamp and the sentiment score, wherein the sharing event record is accessible to the therapist.

9. The method of claim 2, wherein analyzing sentiment further comprises:
detecting neglect indicators comprising:
absence language including “ignored,” “dismissed,” “unavailable,” or “silent”;
emotional void language including “empty,” “alone,” “invisible,” or “disconnected”; and
disengagement language including “didn't respond,” “walked away,” or “shut down”;
when neglect indicators are detected, assigning a sentiment score of zero; and generating a reasoning explanation highlighting the emotional disconnection described in the journal entry. For more complex entries, neglect may be detected along with a score below zero.

10. The method of claim 2, wherein analyzing sentiment further comprises:
detecting abuse indicators comprising:
verbal attack language including “yelled,” “screamed,” “insulted,” or “cursed at”;
contempt language including “mocked,” “ridiculed,” “belittled,” or “degraded”;
control language including “demanded,” “forbade,” “controlled,” or “isolated”;
threat language including “threatened,” “intimidated,” or “menaced”; and
physical harm language including “pushed,” “grabbed,” “hit,” or “hurt”;
assigning severity scores ranging from −1 to −10 based on the intensity and frequency of detected abuse indicators; and
generating a reasoning explanation identifying the specific abusive behaviors detected and their classification on the abuse scale.

11. The method of claim 2, further comprising:
implementing privacy protection by:
storing full journal entry text exclusively on the client mobile device in encrypted local storage;
transmitting only sentiment scores, timestamps, confidence scores, and metadata to the remote server;
configuring the relational database schema to exclude fields for storing journal entry text content; and
providing the therapist with access only to quantitative sentiment data and summary statistics, without access to the qualitative journal entry text unless the client explicitly shares the text during a therapy session.

12. The computer-readable storage medium of claim 3, wherein the instructions further cause the processor to:
implement repair attempt detection by:
maintaining a repair lexicon comprising:
apology patterns including “I'm sorry,” “I apologize,” “forgive me,”or “my mistake”;
reconciliation patterns including “let's talk,” “can we work this out,”“I want to fix this,” or “how can I make it better”;
responsibility patterns including “I was wrong,” “my fault,” “I shouldn't have,” or “I understand why you're upset”;
affirmation patterns including “I love you,” “you matter,” “we'll get through this,” or “I value our relationship”;
classifying the journal entry as containing a repair attempt;
adding +2 to the preliminary sentiment score; and
annotating the reasoning explanation with identification of the specific repair language detected;
scanning the journal entry text for matches to patterns in the repair lexicon; when at least one match is detected:
classifying the journal entry as containing a repair attempt;
adding +2 to the preliminary sentiment score; and
annotating the reasoning explanation with identification of the specific repair language detected.

13. The computer-readable storage medium of claim 3, wherein the instructions further cause the processor to:
implement contextual sentiment adjustment by:
detecting negation terms including “not,” “never,” “no,” “don't,” or “didn't” within a three-word window of sentiment terms;
when a negation term precedes a negative sentiment term, reversing the polarity by converting the negative value to a positive value;
when a negation term precedes a positive sentiment term, reversing the polarity by converting the positive value to a negative value;
detecting intensifier terms including “very,” “extremely,” “really,” “so,” or “completely” within a two-word window of sentiment terms;
when an intensifier term is detected, multiplying the associated sentiment value by a scaling factor ranging from 1.5 to 2.0; and
adjusting the preliminary sentiment score based on the polarity reversals and intensity amplifications. If the entry does not contain indications of care, neglect or abuse, it is considered neutral and not inserted into the database.

14. The system of claim 1, further comprising:
a client onboarding module configured to:
present a tutorial interface to first-time users explaining the Care-Neglect-Abuse continuum model;
provide example journal entries with corresponding sentiment scores for educational purposes;
enable a practice mode wherein clients can enter non-recorded trial journal entries and receive sentiment analysis feedback without storing the entries in the database; and
facilitate therapist-guided initial entry creation by presenting prompts suggested by the therapist during an onboarding therapy session.

15. The system of claim 1, wherein the therapist reporting interface further comprises:
a comparative analysis module configured to:
retrieve event records for both the first client and the second client in a client pair;
generate separate scatter plots for each client displaying their respective sentiment scores over time;
calculate correlation metrics measuring the degree to which sentiment patterns of the first client and second client align or diverge;
identify periods of sentiment divergence where one client's scores trend positively while the other's trend negatively;
highlight periods of sentiment convergence where both clients' scores trend in similar directions, indicating shared emotional experiences; and
display a combined scatter plot and trend line for both client entries.

16. The method of claim 2, further comprising:
conducting validation research by:
enrolling a first cohort of 3-5 trained therapists and their client pairs;
implementing manual sentiment scoring by subject matter experts (SMEs) who review journal entries and independently assign scores on the Care-Neglect-Abuse continuum;
comparing AI-generated sentiment scores to SME-assigned scores to calculate accuracy metrics;
identifying discrepancies where AI and SME scores differ by more than 2 points on the −10 to +10 scale;
refining the sentiment lexicon and natural language processing algorithms based on the identified discrepancies;
enrolling a second cohort of 30+ therapists and 300+ client pairs;
implementing parallel AI and therapist assessment wherein both automated sentiment analysis and therapist clinical judgment evaluate relationship progress; and
validating therapeutic effectiveness by measuring outcome metrics including relationship satisfaction scores, conflict resolution rates, and therapy retention rates.

17. The computer-readable storage medium of claim 3, wherein the instructions further cause the processor to:
calculate the confidence score by:
assigning a base confidence value of 0.5;
increasing the confidence value by 0.1 for each strong lexicon match where a sentiment term appears in the sentiment lexicon with a clear, unambiguous definition;
decreasing the confidence value by 0.1 for each ambiguous term or phrase that could have multiple interpretations;
decreasing the confidence value by 0.2 when sarcasm indicators are detected, including:
contradictions between literal word meaning and likely intended meaning;
exaggerated praise in contexts suggesting negative emotions; or
ironic punctuation patterns such as excessive exclamation marks;
increasing the confidence value by 0.1 when explicit emotion words are present, including “I felt,” “I was,” or “it made me feel”;
capping the confidence value at a maximum of 1.0 and a minimum of 0.0; and
transmitting a low-confidence alert to the therapist reporting interface when the confidence value falls below 0.6, indicating that the journal entry may benefit from human review.

18. The system of claim 1, further comprising:
an adaptation module configured to enable solo therapy mode by:
modifying the client registration process to accept single client accounts not linked to a partner;
configuring the mobile application to present journal entry prompts focused on self-reflection rather than dyadic relationship interactions;
adapting the sentiment analysis engine to analyze personal growth indicators including:
self-compassion language;
insight and awareness statements;
goal-setting and progress descriptions; and
emotional regulation strategies;
generating individual progress reports for the therapist showing trends in self-care, self-criticism, and emotional well-being over time; and
providing the solo client with access to personal sentiment trend visualizations for self-monitoring purposes.

19. The system of claim 1, wherein the AI sentiment analysis engine is implemented using:
a machine learning model trained on a dataset comprising:
annotated relationship journal entries labeled by licensed therapists with sentiment scores on the Care-Neglect-Abuse continuum;
relationship therapy session transcripts with identified instances of care, neglect, abuse, and repair attempts;
validated psychological assessment instruments measuring relationship satisfaction, conflict patterns, and attachment styles; and
wherein the machine learning model comprises one or more of:
a transformer-based natural language processing architecture;
a bidirectional encoder representations from transformers (BERT) model fine-tuned on relationship-specific text;
a long short-term memory (LSTM) recurrent neural network configured to capture temporal dependencies in journal narratives; or
an ensemble model combining outputs from multiple sentiment classification algorithms using weighted voting or stacking methods.
